(19) United States
(12) Patent Application Publication
(10) Pub. No.: US 2025/0276121 A1
(43) Pub. Date: Sep. 4, 2025

(54) DEVICE FOR SUBSTANCE AND/OR HEAT EXCHANGE BETWEEN TWO MEDIA

(71) Applicant: Rheinisch-Westfälische Technische Hochschule (RWTH) Aachen Körperschaft des öffentlichen Rechts, Aachen (DE)

(72) Inventors: Jutta ARENS, Aachen (DE); Franziska SCHUBERT, Aachen (DE); Jan HEYER, Aachen (DE); Thomas SCHMITZ-RODE, Aachen (DE); Ulrich STEINSEIFER, Aachen (DE); Mark SCHOBERER, Aachen (DE); Jannis KLINGE, Aachen (DE); Maximilian LUNEMANN, Aachen (DE)

(21) Appl. No.: 18/859,054

(22) PCT Filed: Jun. 16, 2023
(86) PCT No.: PCT/EP2023/066193

§ 371 Date: Oct. 22, 2024

(30) Foreign Application Priority Data
Jun. 27, 2022 (DE) 10 2022 115 972.8

(51) Int. Cl. A61M 1/36

(52) U.S. Cl. CPC A61M 1/369; A61M 1/3666; A61M 2205/366

(57) ABSTRACT

A device for mass transfer and/or heat exchange between a first medium and a second medium, in particular between blood and a gas or a gas mixture, includes an exchange chamber in which mass-permeable and/or heat-exchanging hollow fibers are disposed, the outside walls of which are sealed at the end regions of the hollow fibers which are spaced apart in the direction of the fiber extension with respect to one another and with respect to a wall region of the exchange chamber and around which the first medium can flow and through which the second medium can flow, wherein the exchange chamber is divided into at least two sub-chambers which each have a sub-volume, mass-permeable and/or heat-exchanging hollow fibers being disposed in each sub-chamber and at least one sub-chamber being movable relative to at least one other sub-chamber and the adjacent sub-volumes of the sub-chambers being connectible to one another or separable from one another by the relative movement of adjacent sub-chambers.

The invention relates to a device for mass transfer and/or heat exchange between a first medium and a second medium, comprising an exchange chamber in which mass-permeable and/or heat-exchanging hollow fibers are disposed, the outside walls of which are sealed at the end regions of the hollow fibers, which are spaced apart in the direction of the fiber extension, with respect to one another and with respect to a wall region of the exchange chamber and around which the first medium can flow and through which the second medium can flow.

Such a device is known from EP 0 183 250 B1, for example.

The first medium can be blood, for example, and the second medium can be a gas or a gas mixture, which preferably contains oxygen. In this use, which is also preferred for the invention, the device forms a so-called oxygenator.

However, the invention is not limited to this use. It is also possible for mass transfer between other media to occur, for example for concentrating or purifying substances in the media. For example, deoxygenation of blood may also be carried out. Likewise, the option exists to introduce gas into a fluid, for example to introduce gas into water.

The exchange, for example a gas exchange and/or heat exchange between the two media, takes place in the exchange chamber. The exchange is carried out through the walls of the hollow fibers, which are permeable with respect to, which is to say, which allow passage of, predetermined substances in the media, for example, allow passage of oxygen and carbon dioxide when used with blood and a gas or gas mixture, in particular by way of diffusion, but do not allow other components of blood, for example blood plasma, to pass.

Such mass-permeable hollow fibers are sufficiently known to a person skilled in the art. Typical hollow fibers that are known from the use of oxygenators are, for example, made of the materials polypropylene (PP), polyethylene (PE), polymethylpentene (PMP) or silicone.

Heat-exchanging fibers shall preferably be understood to mean those that allow energy to be transferred through the walls of the hollow fibers, without additional substances, for example, gas components, being transferred. Heat-transferring fibers are thus preferably not mass-permeable.

The hollow fibers are, for example, disposed in the form of a wound roll of at least one hollow fiber mat in which the hollow fibers are disposed next to one another, for example joined by way of warp threads, wherein the hollow fiber mat is wound around an axis, for example on a core carrying the hollow fibers.

The fibers can also be formed by loose juxtaposition or designed as a laid scrim or a folded product of at least one hollow fiber mat. These types of arrangement are common for use in oxygenators, for example, and are sufficiently known to the person skilled in the art.

By disposing the hollow fibers, at the axial end regions thereof, in a sealed manner with respect one another and with respect to a wall of the exchange chamber, which in technical jargon is also referred to as potting, it is achieved that the exchange chamber is divided into two flow regions by the hollow fiber walls, wherein only the first medium flows in the one flow region and only the second medium flows in the second flow region, such that the two media thus do not come in direct contact with one another, but are separated by the hollow fiber wall, and such that the mass transfer and/or the heat exchange can take place across the hollow fiber wall.

The entire device comprises fluid pathways for feeding the first medium to the one flow region, and for feeding the second medium to the other flow region. The fluid pathways are accordingly likewise separated.

A fluid pathway, and in particular an inlet and an outlet for the first medium, for example blood, which contacts the hollow fiber outside walls, can, for example, lead in each case through an outside wall of the exchange chamber into the same.

So as to guide the second medium, preferably a gas or a gas mixture, through the fibers, the exchange chamber is surrounded by two media compartments, in particular gas compartments, in the flow direction of the second medium, which adjoin the exchange chamber in a sealed manner and which are connected to the interior of the hollow fibers, wherein in particular the axial open ends of which preferably open into the media compartments/gas compartments through the seal/potting of the hollow fibers with respect to one another and with respect to at least one wall of the exchange chamber.

It has proven to be disadvantageous with such mass transfer exchange devices, in particular when designed as oxygenators, for the exchange surface and the volume for the media in the device to be constant and such that they cannot be adapted to changing conditions during use.

When the usage conditions change, a previously used mass transfer device would thus have to be replaced with another having a different volume or a different exchange surface. However, this is not possible in all applications.

The problem that arises with extracorporeal oxygenation of premature babies is, for example, of the development of the premature baby being accompanied by a rapid increase in blood volume, but an oxygenating mass transfer device cannot be readily exchanged for a larger device, in particular as this would jeopardize the supply to the premature baby.

Furthermore, it is difficult to wean individuals who receive supportive treatment from an oxygenator when using the known mass transfer devices.

Against this background, it is an object of the invention to provide mass transfer devices of the type mentioned at the outset, in particular oxygenators, which make it possible to change the volume of at least one of the media involved in the exchange, in particular blood, and/or the exchange surface during operation without replacing the entire device, which is to say, without removing the device from use and replacing it with another. In particular, it is to be achieved that the volumes of the media in the device and/or the exchange surface can be increased and/or decreased.

According to the invention, this object is achieved by dividing the exchange chamber into at least two sub-chambers which each have a sub-volume, wherein mass-permeable and/or heat-exchanging hollow fibers are disposed in each sub-chamber and at least one sub-chamber can be moved relative to at least one other sub-chamber, wherein the adjacent sub-volumes of the sub-chambers can be connected to one another or separated from one another by the relative movement of adjacent sub-chambers.

In conjunction with the sub-volumes and the hollow fibers disposed in the sub-chambers, this means that the usable exchange surface also increases when the usable total volume is increased due to the connection of at least two sub-chambers, or that the usable exchange surface also decreases when the usable total volume decreases when at least two sub-chambers are separated.

The sum of the sub-volumes of all sub-chambers forms the maximum possible total volume of the exchange chamber, wherein in particular the minimum possible volume is defined by the volume of a single sub-chamber through which flow is possible.

With the aforementioned generally sealed arrangement of the hollow fibers in the exchange chamber, in an embodiment according to the invention of the division of the exchange chamber into multiple sub-chambers, in each of the sub-chambers, the outside walls of the hollow fibers of the relevant sub-chamber are sealed at the end regions of the hollow fibers, which are spaced apart in the direction of the fiber extension, with respect to one another and with respect to a wall region of the particular sub-chamber, which in particular corresponds to the potting mentioned at the outset.

The seal can preferably be at least achieved by a potting agent, for example polyurethane or silicone, possibly in conjunction with at least one further sealing element.

The invention thus allows for the possibility of selectively increasing or decreasing the usable volume and/or the usable exchange surface of the hollow fibers by connecting or separating at least two sub-chambers of the exchange chamber, in particular during ongoing use, for example oxygenation of a patient.

As a result, it is possible to respond to the increase in blood during the development of premature babies by increasing the utilized volume and/or the utilized exchange surface of the device by connecting a previously not utilized sub-chamber to at least one previously utilized sub-chamber.

In contrast, the procedure for weaning patients off artificial oxygenation may be such that at least one sub-chamber of at least two previously utilized sub-chambers is separated so as to decrease the volume and/or the exchange surface.

According to the invention, the particular change is carried out by moving two sub-chambers, in particular adjacent sub-chambers, relative to one another in the device, wherein, depending on the relative position assumed by these sub-chambers, the two adjacent sub-chambers are connected to one another or separated from one another.

The invention may provide that the connection or separation of two sub-chambers, and the attendant increase in volume, only have an effect in terms of the volume of one of the two media, for example, only the volume of the blood, and/or in terms of the exchange surface contacting one of the two media, for example, only the blood.

This means that the volume in the device which is taken up by the other medium, for example gas/gas mixture, or the exchange surface that is contacted by the other medium, for example gas/gas mixture, remains the same in both positions.

During use, one of the two media can thus simultaneously flow through each sub-chamber in the entire device, regardless of the set position, and in particular flows through the flow region of all sub-chambers through the interior of the hollow fibers, and preferably the flow region through which the gas/gas mixture flows during use of the oxygenator.

In this preferred embodiment, the connectible and separable sub-volumes of the sub-chambers are thus sub-volumes that can only be taken up by one of the two media. However, the invention can also provide that the sub-volumes of the two media in the sub-chambers can be connected and separated.

So as to achieve the relative movement of two adjacent sub-chambers with respect to one another, the invention provides movably mounting at least one sub-chamber of at least two sub-chambers in the device.

According to a preferred embodiment, it may be provided that adjacent sub-chambers form a juxtaposition. In particular, adjacent sub-chambers can each be disposed completely next to one another, on each side of a separation plane.

In the case of juxtaposition, the sub-chambers can all have parallel longitudinal extension directions, which in particular are at least substantially parallel to the extension directions of the hollow fibers.

Another preferred embodiment can provide that adjacent sub-chambers form an inside-one-another arrangement, in particular that at least one inner sub-chamber is surrounded by at least one outer sub-chamber.

Regardless of the type of arrangement of the sub-chambers, it may preferably be provided that opposing wall regions of adjacent sub-chambers bear on one another in a sealed manner and each has wall passages that can be brought into a first position by the relative movement, in which the wall passages at least partially overlap one another, and can be brought into a second position, in which the wall passages do not overlap one another.

In the overlapping position, a medium, for example blood, can thus cross between the sub-chambers through the at least regionally, and preferably fully, overlapping wall passages. In the non-overlapping position, in contrast, a wall region of the adjacent sub-chamber overlaps a wall passage in one of the sub-chambers, such that media is not able to pass through.

The sealed bearing of the opposing wall regions of adjacent sub-chambers on one another can, for example, be achieved by a technical zero gap between the wall regions, which allows a movement between the wall regions, but does not allow either of the media to penetrate the zero gap or, for example, by at least one sealing element, which acts between the wall regions and prevents the medium from being able to penetrate the gap between the wall regions.

A technical zero gap can, for example, be designed to be so small that blood components, in particular blood plasma, cannot penetrate the gap.

Generally speaking, it may be provided that at least one sub-chamber can be displaced with respect to at least one adjacent sub-chamber along a predetermined direction, for example a longitudinal extension direction of the sub-chamber(s), and/or that a sub-chamber can be rotated, in particular about an axis of rotation.

In the case of the aforementioned juxtaposition, for example, the sub-chambers can be made displaceable relative to one another. In the case of the aforementioned inside-one-another arrangement, for example, a sub-chamber can be made displaceable and/or rotatable with respect to another adjacent sub-chamber.

In a preferred embodiment, it is provided that adjacent sub-chambers, and preferably all sub-chambers of the device, are disposed around a shared axis. This shared axis can, for example, be situated parallel to the longitudinal extension direction of the sub-chamber(s), and in particular can coincide with the flow direction of one of the two media, for example the media flow direction/gas flow direction that leads through the hollow fibers.

In this embodiment, at least one sub-chamber can be displaced with respect to at least one adjacent sub-chamber along the shared axis and/or can be rotated about the shared axis.

Preferably, each sub-chamber is formed by an outside wall surrounding the shared axis and an inside wall disposed radially spaced apart therefrom and surrounding the shared axis, wherein the mass-permeable and/or heat-exchanging hollow fibers of the sub-chamber are disposed in the spaced-apart region between the outside wall and inside wall, which in axially spaced-apart end regions are sealed with respect to one another and with respect to the outside wall and the inside wall of the sub-chamber.

Such a sealed arrangement can be effected by way of a bond, in particular by a bond alone or a combination of a bond and an elastomeric sealing element. The bond itself can be formed by a liquid sealant that is applied and then cured, which in the cured state forms an elastomer and which either acts sealingly alone or acts in conjunction with another elastomeric sealing element.

More preferably, it is provided that adjacent sub-chambers in each case have at least two, and preferably exactly two, wall passages through which the sub-volumes of the adjacent sub-chambers can be connected. In particular, adjacent shall be understood to mean that the wall regions of adjacent sub-chambers abut one another in the radial direction, which is to say that preferably an inside wall of an outer sub-chamber and an outside wall of an inner sub-chamber are located opposite one another.

As a result of the arrangement of the inside wall and the outside wall of a sub-chamber, having the hollow fibers disposed therebetween, the particular sub-chamber can form a cylindrical arrangement. This arrangement can be hollow-cylindrical, in particular if a further sub-chamber is located in this hollow-cylindrical arrangement.

The cross-sectional shape perpendicular to the cylinder axis of the cylindrical, in particular hollow-cylindrical, arrangement of a sub-chamber can generally be arbitrary, provided the relative movement of the sub-chambers with respect to one another allows displacement along the cylinder axis or the shared axis. If the relative movement allows rotation, the cross-sectional shape of the arrangement of a sub-chamber or of the outside wall and inside wall thereof is circular cylindrical, in particular in all sub-chambers or at least in two adjacent sub-chambers.

The invention can preferably provide that the at least one, and preferably exactly one, wall passage of each sub-chamber through which the first medium can flow into the sub-chamber is disposed at a different axial height than the at least one, and preferably exactly one, wall passage through which the first medium can flow out of the sub-chamber. In this way, it is achieved, that the particular sub-chamber through which flow occurs can be flown through in a direction of the axial spacings of the wall passages.

More preferably, it is provided, in particular in conjunction with the aforementioned embodiment, that the at least one wall passage through which the first medium can flow into the sub-chamber and the at least one wall passage through which the first medium can flow out of the sub-chamber are disposed opposite one another around the shared axis, preferably at an angular distance of 180 degrees. In this way, diametrical flow through the sub-chambers is achieved, in particular in conjunction with the aforementioned embodiment.

In a preferred embodiment, the invention can furthermore provide that wall passages that are assigned to one another or the mouth openings, facing the interior of a sub-chamber, of adjacent sub-chambers, through which the first medium can cross between the adjacent sub-chamber, are situated at the same axial height, in particular extend across the same axial height region, and preferably extend across the same angular range around the shared axis. Wall passages are, in particular, assigned to one another when these cooperate for the purpose of allowing or preventing media transfer between the adjacent sub-chambers.

Viewed in the axial direction, moreover, each wall passage, or at least the mouth opening of each wall passage of a sub-chamber which faces the interior of a sub-chamber, can preferably be surrounded by sealing regions in which mutually facing walls of the adjacent sub-chambers bear on one another in a sealed manner.

A sealing region is formed, for example, in each case, by a sealing ring that is recessed in an annular groove on only one of the two mutually facing walls.

Preferably, it is provided that the wall region of the outside wall and/or of the inside wall which faces the interior of a sub-chamber comprises several protrusions around the shared axis in the circumferential direction, which in particular extend unvaryingly n the axial direction.

Such protrusions can be used to cause medium flowing close to the edge on the wall to be redirected radially inwardly into the region of the hollow fibers.

The curvature/the progression of the wall region, viewed in the cross-section perpendicular to the shared axis, is preferably different on each side of a particular protrusion.

For all possible embodiments, the invention can preferably provide that the flow resistances for the first medium are identical in adjacent sub-chambers, and preferably in each sub-chamber, in particular other than an inaccuracy of plus/minus 30%, preferably plus/minus 20%, preferably plus/minus 10%, preferably plus/minus 5%. Furthermore, the sub-volumes of adjacent sub-chambers, and preferably of each sub-chamber, can be identical, in particular other than an inaccuracy of plus/minus 30%, preferably plus/minus 20%, preferably plus/minus 10%, preferably plus/minus 5%.

These embodiments preferably cause the flow conditions in each of the connected sub-chambers to be identical or at least similar when the sub-chambers are connected so that equivalent flow through connected sub-chambers is at least substantially possible.

According to a preferred refinement, the mouth opening of a wall passage opening into the interior of a sub-chamber, in particular at least in the sub-chamber located radially the furthest to the outside, and preferably only in the sub-chamber located radially the furthest to the outside in the circumferential direction around the shared axis, preferably in the two opposing circumferential directions around the shared axis, transitions into a groove that is open toward the interior of the sub-chamber, in particular the angular extension of which around the shared axis is larger than the angular extension of the through-opening extending through the entire wall thickness. As a result, the medium flowing into the sub-chamber through this wall passage can be easily distributed around the hollow fibers in the circumferential direction.

In the sub-chamber located radially the furthest to the outside, such a wall passage can open into a connector through which the medium, for example blood, can be fed into the device and/or be discharged therefrom.

Furthermore, it may be provided, in particular for optimizing flow guidance, that the wall passage, in particular the center thereof, which preferably forms an outlet for the first medium out of the sub-chamber, preferably out of the sub-chamber located the furthest to the outside, is axially offset vertically in relation to the mouth opening and/or the groove that is open toward the interior of the sub-chamber, and in particular is offset downwardly in the intended usage position.

The outside wall of the sub-chamber of the device which is located radially the furthest to the outside preferably forms the outer housing wall or at least a part of the outer housing wall of the entire device.

The axial ends preferably comprise connecting means, preferably threaded regions. These are, for example, suitable for the connection to wall regions of the device which enclose the gas compartments of the device through which the gas/gas mixture can be conducted to/from the interior of the hollow fibers, or for the connection to a connecting piece so as to implement a heat exchanger function in one of several sub-chambers and to be able to introduce fluid required for this purpose into the interior of the hollow fibers of this sub-chamber.

More preferably, the invention can provide, in all possible variant embodiments, that one of the two walls, these being the inside wall and the outside wall, which delimit a sub-chamber is guided in the axial direction in a sealed manner through a gas compartment of the device and a wall region of the device which delimits the gas compartment to the outside, wherein a handle is attached, or at least can be attached, to the guided-through region, by way of which the sub-chamber can be rotated about the shared axis or can be displaced along the shared axis. Such a handle can, for example, be attached to or inserted into the guided-through wall in a form-locked manner.

Such a guided-through wall can, for example, be the inside wall of an innermost sub-chamber of an arrangement of sub-chambers in which these are disposed inside one another.

This inside wall of the sub-chamber located radially the furthest to the inside can preferably form a core of the device, which is guided in the axial direction in a sealed manner through a wall region of the device which delimits a gas compartment, wherein a handle is attached, or at least can be attached, to the guided-through core, for example by way of form-locked attachment, by which the sub-chamber can be rotated about the shared axis or can be displaced along the shared axis.

All sub-chambers can be disposed around the core, and the shared axis can, in particular, extend, preferably centrally, through the core. This inside wall or the core can rest rotatably and/or displaceably in a respective bearing region of the two gas compartments or, generally speaking, of the two media compartments which are used to feed medium into the interior of the hollow fibers.

A preferred embodiment of the invention comprises exactly two sub-chambers. The inner sub-chamber can be moved, preferably rotated and/or axially displaced, relative to the outer sub-chamber, which is in particular enclosed by the housing outside wall.

A contact region of an outside wall and/or inside wall of a sub-chamber in which the wall contacts a potting compound can preferably be coated with an adhesion promoter or can be structured differently than a contact region of the wall in which the first medium is contacted. This different structuring can be formed by a roughened region or by a region that is provided with grooves or recesses.

A region that is provided with grooves or recesses can be formed at a separate, preferably elastomeric component, which formed in an annular recessed region, preferably at an axial end of an outside wall and/or inside wall of a sub-chamber. This component can form a hollow sleeve, which has grooves or recesses on the surface region that faces the interior of the sub-chamber, into which potting compound can penetrate during potting or has penetrated after potting. Grooves or recesses can extend 360 degrees around the shared axis in the described surface region. Several grooves or recesses can be disposed axially next to one another.

In all possible embodiments, it may be provided that mass-permeable hollow fibers are disposed in at least one of several sub-chambers, and that heat-exchanging hollow fibers are disposed in at least one of several sub-chambers. The creation of a heat exchanger function can thus be implemented in at least one of several sub-chambers. This heat-exchanging sub-chamber can be subject to a flow of a third medium, which in particular flows through the hollow fibers on the inside.

Various media which are fed to different sub-chambers so as to flow through the interior of each hollow fiber, can be fed through various fluid infeeds, which are disposed in different radial positions relative to the shared axis and which open into the interior of each hollow fiber of different sub-chambers.

As mentioned at the outset, the device according to the invention preferably forms an oxygenator for enriching blood, serving as the first medium, with oxygen, and/or for depleting carbon dioxide from blood, serving as the first medium. The device comprises hollow fibers that are suited for this function in at least some of all the sub-chambers. The second medium is a gas or gas mixture which supplies oxygen to the blood and removes carbon dioxide from the blood. If necessary, a third medium can be provided for the heat exchange.

Exemplary embodiments of the invention will be described based on the figures.

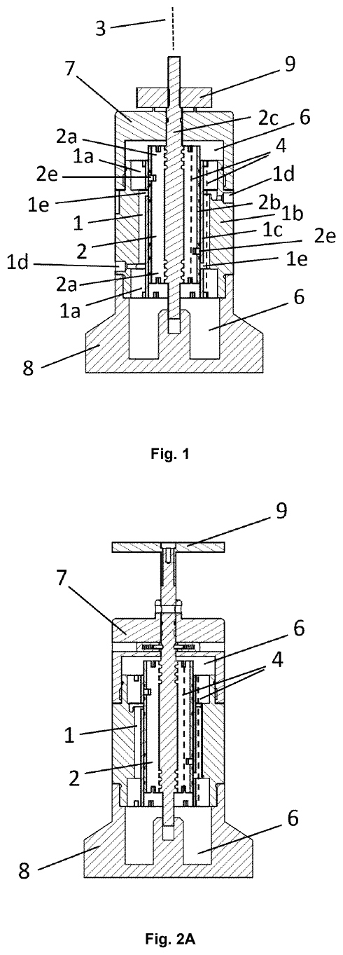

FIG. 1 shows a first specific embodiment of a device according to the invention comprising two sub-chambers that can be axially displaced relative to one another;

FIG. 2 show a second specific embodiment of a device according to the invention comprising two sub-chambers that can be axially displaced relative to one another with details being enlarged;

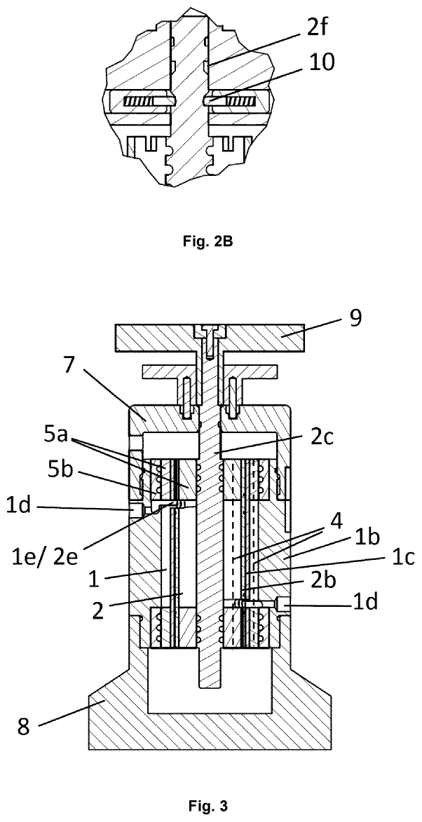

FIG. 3 shows a specific embodiment of a device according to the invention comprising two sub-chambers that can be rotated relative to one another about a shared axis;

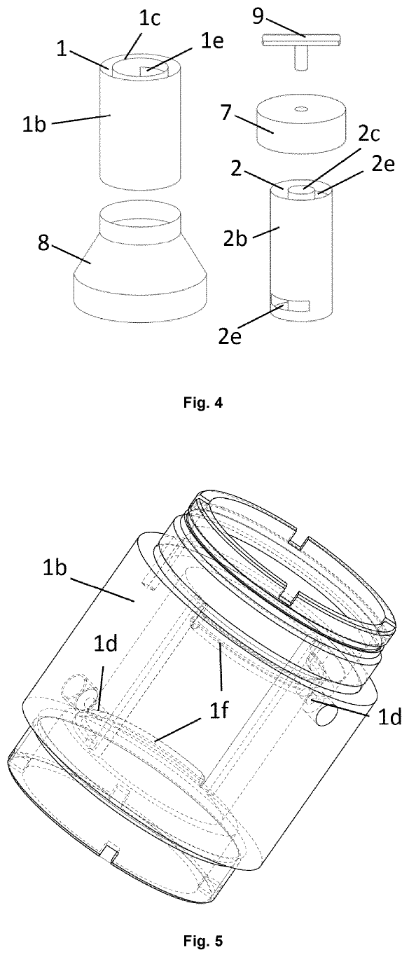

FIG. 4 shows a simplified illustration of the embodiment according to FIG. 3 in separate illustrations of the individual elements;

FIG. 5 shows the outside wall of the radially outer sub-chamber of the device according to FIGS. 3 and 4, which also forms a part of the housing of the device;

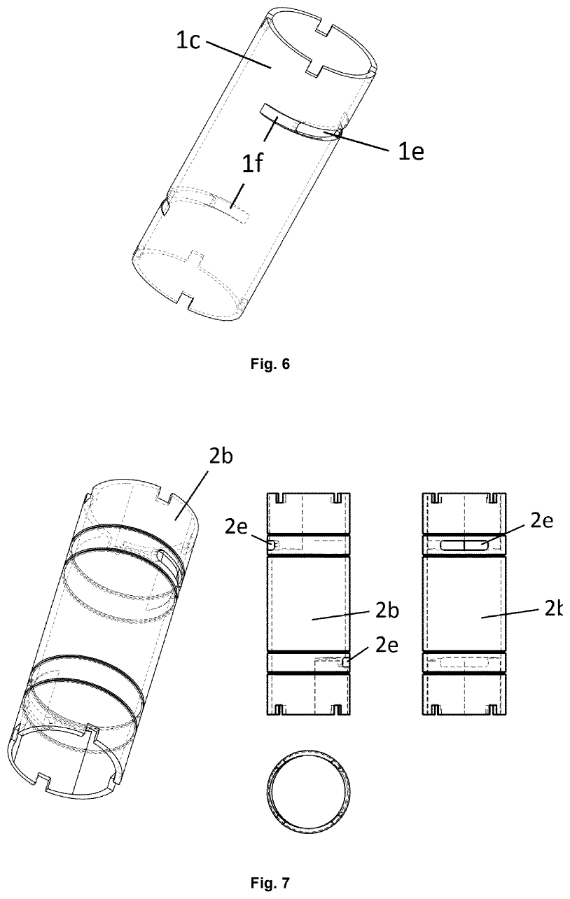

FIG. 6 shows the inside wall of the radially outer sub-chamber of the device according to FIGS. 3 and 4;

FIG. 7 shows several views of the outside wall of the radially inner sub-chamber of the device according to FIGS. 3 and 4;

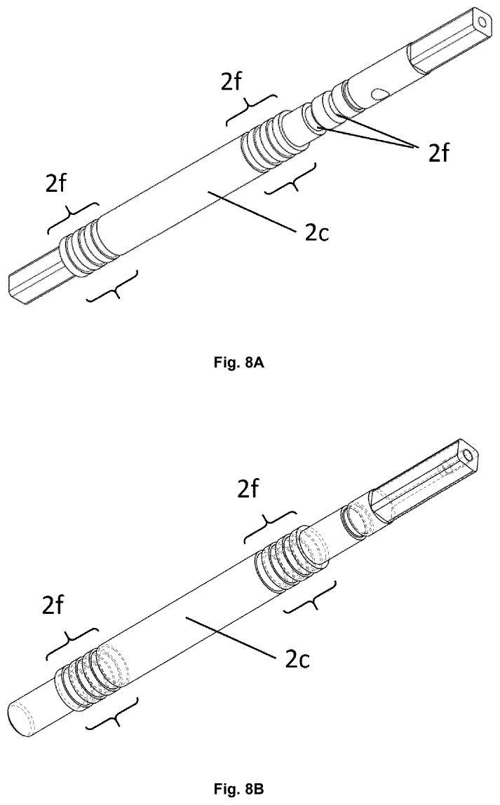

FIG. 8 show core elements of the first and second embodiments of the devices according to the invention, the outer surfaces of which form the inside wall of the respective radially inner sub-chamber;

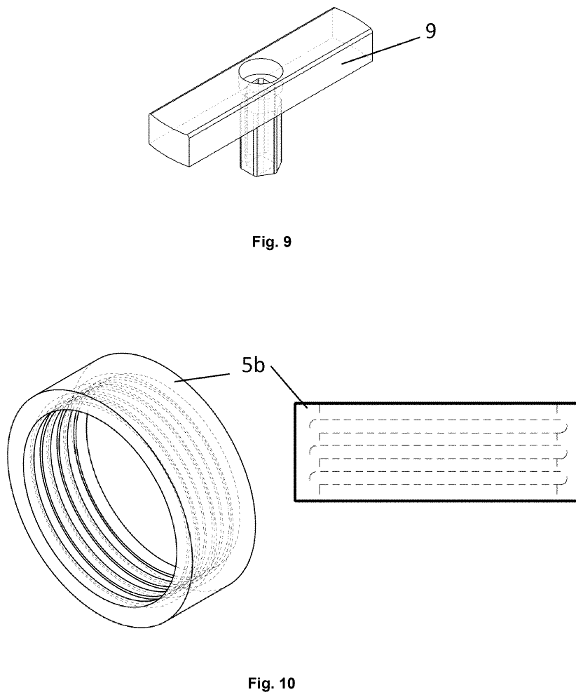

FIG. 9 shows a handle that can be connected to the respective core elements of FIG. 8 for manually carrying out the relative movement of the sub-chambers;

FIG. 10 shows a sealing element including an insert in conjunction with potting agent at the axial end regions of the outside wall of the outer sub-chamber; and

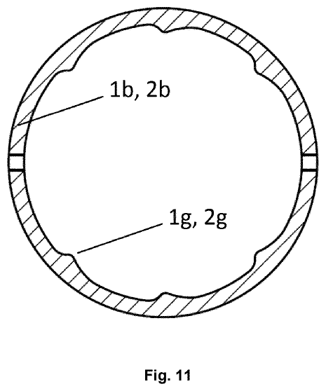

FIG. 11 shows a possible design of the inner surface of an outside wall of a sub-chamber in an axial cross-section.

FIG. 1 shows a first specific embodiment of the device according to the invention. In this embodiment as well as in the embodiments shown and described hereafter, the entire exchange chamber of the device is divided into two sub-chambers 1 and 2. The sub-chamber 1 is situated radially on the outside, and the sub-chamber 2 is situated radially on the inside, with respect to an axis 3 which preferably corresponds to the longitudinal axis of the sub-chambers 1, 2 and the entire device. The sub-chamber 2 is thus surrounded by the sub-chamber 1. Both are situated coaxial to the center axis 3.

On the right side of FIG. 1, the position of hollow fibers 4 in each of the two sub-chambers 1, 2 is indicated by way of example by a dotted line. The hollow fibers 4 are sealed at the axial ends thereof with respect to one another and with respect to the walls of the sub-chambers 1, 2. A potting agent suitable for this purpose, potentially together with a sealing element, is accommodated in axial end regions 1a of sub-chamber 1 and 2a of sub-chamber 2 intended for this purpose.

The potting agent 5a, and possibly a sealing element 5b, are not visualized in the illustration of FIGS. 1 and 2 and are shown hatched in FIG. 3. The potting agent 5a can thus preferably also indirectly contact a wall of a sub-chamber, here preferably at least the outside wall of the radially outer sub-chamber 1, via a sealing element 5b having a structured surface.

The outside wall 1b of the radially outer sub-chamber 1 at the same time also forms a wall of the entire housing of the device. The inside wall 1c of the radially outer sub-chamber 1 is designed as a hollow-cylindrical element, in particular having a circular cross-section. The outside wall 2b of the radially inner sub-chamber 2 has a similar design. The inside wall 2c of the radially inner sub-chamber 2, in contrast, forms a core element of the device through which the axis 3 runs.

Through the wall passages 1d of the radially outer sub-chamber 1, a medium, for example blood, can be guided through the sub-chamber 1. The wall passages 1d are preferably spaced 180 degrees apart around the axis 3 and disposed in different axial positions, in particular in each case before the start of the potting. This achieves a diametrical flow through the sub-chamber 1, for example when flow only occurs through this sub-chamber.

The medium contacts the outer surface of the hollow fibers 4. A gas or gas mixture can be guided through the interior of the hollow fibers 4. For this purpose, the open axial ends of the hollow fibers extend axially on both sides into gas compartments 6. Corresponding ports for supplying and removing gas or a gas mixture are provided in the cover element 7 and the base 8 of the device, but are not visualized. The cover element 7 and the base 8 each adjoin the axial end regions of the outside wall 1b of the outer sub-chamber 1 in a sealed manner. Each of these can, for example, be connected via a thread.

The outer sub-chamber 1 has two further wall passages 1e in the inside wall 1c, which are preferably disposed opposite one another around the axis 3, but in different axial positions, in particular, specifically, in each case before the start of the axially end-side potting 5.

The inner sub-chamber 2 has an assigned wall passage 2e for each wall passage 1e. When the wall passages 1e and 2e are disposed so as to overlap one another as a result of the relative positioning of the two sub-chambers 1, 2, the medium can cross between the sub-chambers 1, 2, but not when the wall passages 1e, 2e are not disposed so as to overlap. Due to a relative movement of the two sub-chambers between two defined positions, which define an overlapping position and a non-overlapping position of the wall passages 1e, 2e, the sub-volume and the exchange surface of the adjacent sub-chambers 1 and 2 can thus either be interconnected or separated from one another.

So as to be able to carry out the relative movement between these positions, the inside wall 2c of the inner chamber 2, which is the core element here, is guided, sealed by the upper gas compartment 5 and the cover 7, to the outside and comprises a handle 9 for actuation, which is accessible from the outside on the device. In the cover 7 and the base 8, the core element or the inside wall 2c is accordingly movably mounted so as to be able to switch between the two positions.

The sub-chambers 1, 2 or the walls thereof preferably have the same axial lengths.

The aforementioned description of FIG. 1 relates to design details, which are preferably present in all shown and/or also in not shown embodiments of the invention, which is to say in particular regardless of the type of the relative movement of the adjacent sub-chambers with respect to one another. The details preferably also apply to the embodiments of the following figures, in particular in conjunction with designs additionally described for these figures.

In the embodiment of FIG. 1, it is provided that the inner sub-chamber 2 can be displaced axially parallel to the axis 3 relative to the outer sub-chamber 1, in particular without rotatability about the axis 3. FIG. 1 specifically shows an axially displaced position of the inner sub-chamber 2 with respect to the outer sub-chamber 1, which is in particular apparent from the fact that the axial ends of the sub-chambers are located in different axial positions.

In FIG. 1, the wall passages 1e and 2e of the sub-chambers 1, 2 are located in different axial positions, which is to say do not overlap one another, so that media transfer through the two sub-chambers is prevented. As a result, only the sub-volume and the exchange surface of the sub-chamber 1 act in this position.

If, in contrast, the sub-chamber 2 is moved axially downward, for example as a result of the actuation of the handle 9, the wall passages 1e, 2e overlap one another, and the sub-volume and the exchange surface of the sub-chambers 1, 2 are interconnected.

For the actuation, it may be provided in the embodiment of FIG. 1 that a turning wheel forms the handle 9, which is rotatably mounted at the cover 7 so as to be axially stationary and has a threaded connection to the part of the core element or of the inside wall 2c located on the outside, so that the axial displacement of the sub-chamber 2 is carried out by way of rotation.

FIG. 2 shows a second embodiment with an axial relative displacement of the sub-chambers 1 and 2 with respect to one another. Differing from FIG. 1, the handle 9 is designed as a grip, which is being axially displaced. Two detent positions are implemented, which correspond to the overlapping and the non-overlapping positions of the wall passages 1e and 2e.

For this purpose, the core element or the inside wall 2c has detent recesses 2f at a detent region, in which a, preferably spring-loaded, detent element 10, which can be implemented, for example, in or at the cover 7, can engage so as to define the detent positions.

In the embodiments of FIGS. 1 and 2, the inside wall 2c or the core element is in each case mounted so as to be axially displaceable in the cover 7 and the base.

Compared to FIGS. 1 and 2, the embodiment of FIG. 3, in particular with otherwise identical features, shows a relative movability of the sub-chambers 1, 2 by a rotation of the sub-chambers 1, 2 relative to one another about the shared axis 3. In this case, the inside wall 2c of the inner sub-chamber 2 or the core element is mounted rotatably in the cover 7 and the base 8.

The cross-sectional shape perpendicular to the axis 3 of the inside wall 1c of the outer sub-chamber 1 and the outside wall 2c of the inner sub-chamber 2 has a circular ring-shaped design here. In axially displaceable embodiments, this cross-sectional shape, in contrast, can be hollow-cylindrical having an otherwise arbitrary geometry.

FIG. 4 shows a simplified view of this specific embodiment of FIG. 3 with elements that are separated from one another, without the hollow fibers being shown. In the selected illustration, the wall passages 1e, 2e of the sub-chambers 1, 2 are located in an overlapping position and can be brought into a non-overlapping position by rotation with respect to one another.

FIG. 5 shows a detailed illustration of the outside wall 1b of the radially outer sub-chamber 1, and FIG. 6 shows the associated inside wall 1c.

What is essential is that, in both figures, the mouth opening of the wall passage 1d or 1e which opens into the interior of the sub-chamber 1 transitions in the circumferential direction around the shared axis 3, preferably in the two opposite circumferential directions around the shared axis 3, into a groove 1f that is open toward the interior of the sub-chamber. The angular extension thereof around the shared axis 3 is preferably larger than the angular extension of the through-opening extending through the entire wall thickness or than the mouth opening on the opposite side of the wall passage.

FIG. 7 shows the outside wall 2b of the inner sub-chamber 2 including the wall passages 2e, which are assigned to the wall passages 1e so as to functionally cooperate therewith.

So as to achieve sealing between the inside wall 1c and the outside wall 2b of the two sub-chambers in the axial direction, two axially spaced-apart grooves are provided on one of the surfaces of the two walls which face one another, which axially surround the wall passage, which here is the wall passage 2e, and in which sealing rings are recessed. The opposing surfaces of the inside wall 1c and outside wall 2b thus bear on one another in a sealed manner by way of these sealing rings. FIG. 7 shows the seal in the variant that can be rotated about the axis.

In the axially displaceable variant, preferably three axially spaced-apart grooves, each having sealing rings, are provided, wherein each wall passage is located in an axial intermediate space between two sealing rings. In the overlapping arrangement of the wall passages, the two wall passages are located between the same axial intermediate space, and in the non-overlapping position, the wall passages are each located in various axial intermediate spaces. In FIG. 2A, the position of the sealing rings/of the grooves is indicated for the axially displaceable variant.

FIG. 8A shows the inside wall 2c or the core element for the axially displaceable variant of FIG. 2, and FIG. 8B shows this for the rotatable variant. Due to the polygonal lower axial end, the core element of FIG. 8A is not rotatable, but is axially displaceable. In an axial region between the upper potting and the attachment region of the handle, the core element comprises the above-described detent region including the annular detent recesses 2f.

This can be dispensed with in the rotatable variant of FIG. 8B. An annular groove for receiving a sealing ring can be provided in both movement variants, axially beneath the attachment region for the handle.

It is preferably provided in this embodiment for both variants that the potting region 2g which comes in contact with the potting agent is structured. The structuring here is formed by at least two axially spaced-apart annular recesses on the surface of the inside wall 2c or of the core element which faces the interior of the sub-chamber.

FIG. 9 shows a handle, for example for the embodiment of FIGS. 2 and 3. This handle can be attached in a form-locked manner to the attachment region of the inside walls 2c or of the particular core element. For example, the handle has an axially extending recess, which deviates from a circular cross-section and here, for example, is polygonal, for receiving a correspondingly designed attachment region in a form-locked manner at the inside wall 2c.

FIG. 10 shows two views of a sealing element 5b, which can be used together with potting compound so as to seal the hollow fibers 4 with respect one another and with respect to the wall of the sub-chamber. With the radially outer surface, the annular sealing element 5b shown here directly contacts the surface of the outside wall of the inner sub-chamber 1, and with the structured radially inner surface, the annular sealing element contacts the potting compound. The structuring here is formed by at least two axially spaced-apart annular recesses on the surface of the sealing element which faces the interior of the sub-chamber. At the axial end side, the outside wall of the sub-chamber 1 can include a region having an increased inside diameter for accommodating the sealing element.

FIG. 10 shows a design of the inner shape of the outside wall of the sub-chamber 1 or of the sub-chamber 2. What is essential here is that the surface of the outside wall facing the interior of the sub-chamber 1 or 2 comprises several protrusions 1g/2g. These ensure that there is no edge flow that leads around the hollow fibers in the interior. The protrusions always direct the medium radially to the inside toward the hollow fibers. Corresponding protrusions can also be provided at the surface of the inside wall of the sub-chambers 1 and 2 which face the interior.

The shown cross-sectional shape of the outside wall 1b, 2b is unvarying in the axial direction. Furthermore, the curvature/the progression of the wall region, viewed in the cross-section perpendicular to the shared axis 3, is preferably different on each side of a particular protrusion 1g, 2g.

The shown devices can be used as oxygenators, for example, in which, during treatment of a patient, the acting exchange surface and the acting volume can be changed by moving the two sub-chambers 1 and 2 relative to one another, in particular so as to switch the sub-chambers 1, 2 between two defined positions.

The project that resulted in this application was financed by the Horizon 2020 Research and Innovation program of the European Union according to grant agreement no. 863087.

1. A device for mass transfer and/or heat exchange between blood as a first medium and a gas or a gas mixture as a second medium, comprising an exchange chamber in which mass-permeable and/or heat-exchanging hollow fibers are disposed, the outside walls of which are sealed at the end regions of the hollow fibers which are spaced apart in the direction of the fiber extension with respect one another and with respect to a wall region of the exchange chamber and around which the first medium can flow and through which the second medium can flow, wherein the exchange chamber is divided into at least two sub-chambers which are adjacent to one another and each have a sub-volume adjacent to the sub-volume of at least one other of the sub-chambers, the mass-permeable and/or heat-exchanging hollow fibers being disposed in each of the sub-chambers and at least one of the sub-chambers being movable relative to at least one other of the sub-chambers and the adjacent sub-volumes of the sub-chambers being connectible to one another or separable from one another by the relative movement of the adjacent sub-chambers.

2. The device according to claim 1, wherein the adjacent sub-chambers
a. the adjacent sub-chambers are each disposed completely next to one another on each side of a separation plane; or
b. form an inside-one-another arrangement in which at least one of the sub-chambers is an inner sub-chamber surrounded by at least one other of the sub-chambers which is an outer sub-chamber.

3. The device according to claim 1, wherein opposing wall regions of the adjacent sub-chambers bear on one another in a sealed manner and each has wall passages that can be brought into a first position by the relative movement, in which first position the wall passages at least partially overlap one another, and can be brought into a second position, in which second position the wall passages do not overlap one another.

4. The device according to claim 1, wherein all of the adjacent sub-chambers are disposed around a shared axis and at least one of the sub-chambers can be displaced with respect to at least one of the adjacent thereto along the shared axis and/or can be rotated about the shared axis.

5. The device according to claim 4, wherein each of the sub-chambers is formed by an outside wall surrounding the shared axis and an inside wall spaced radially apart therefrom and surrounding the shared axis, the mass-permeable and/or heat-exchanging hollow fibers of each of the sub-chambers being disposed in a region between the radially spaced-apart outside and inside walls, which in axially spaced-apart end regions are sealed against one another and with respect to the outside wall and the inside wall of each of the sub-chambers.

6. The device according to claim 5, wherein the adjacent sub-chambers each comprise only two wall passages through which the sub-volumes of the adjacent sub-chambers can be connected.

7. The device according to claim 6, wherein each of the sub-chambers has only one wall passage through which the first medium can flow into one of the sub-chambers and that one wall passage is disposed at a different axial height than an only one wall passage through which the first medium can flow out of the sub-chamber.

8. The device according to claim 7, wherein the one wall passage through which the first medium can flow into the sub-chamber and the one wall passage through which the first medium can flow out of the sub-chamber are disposed opposite one another around the shared axis, at an angular distance of 180 degrees.

9. The device according to claim 6, wherein wall passages or mouth openings of the wall passages facing an interior of a sub-chamber of the adjacent sub-chambers through which the first medium can cross between the adjacent sub-chambers extend across a same axial height region and across a same angular range around the shared axis.

10. The device according to claim 9, wherein, viewed in an axial direction, each of the wall passages or the mouth opening of each of the wall passages of the sub-chamber which faces the interior of the adjacent sub-chamber is surrounded by sealing regions in which the walls of the adjacent sub-chambers which face one another bear on one another in a sealed manner, a sealing region being formed in each case by a seal that is recessed in an annular groove on only one of the two walls facing one another.

11. The device according to claim 10, wherein the wall region of the outside wall and/or inside wall which faces the interior of a sub-chamber in the circumferential direction comprises a plurality of protrusions around the shared axis which extend unvaryingly in the axial direction, the curvature or progression of the wall region, viewed in the cross-section perpendicular to the shared axis in each case being different on each side of each of the protrusions.

12. The device according to claim 11, wherein flow resistances for the first medium in each of the adjacent sub-chambers are identical other than an inaccuracy of plus/minus 30%.

13. The device according to claim 12, wherein the sub-volumes of each of the adjacent sub-chambers are identical other than an inaccuracy of plus/minus 30%.

14. The device according to claim 11, wherein the mouth opening of the wall passage opening into the interior of only the sub-chamber located radially the furthest to the outside in the two opposing circumferential directions around the shared axis transitions into a groove that is open toward the interior of the sub-chamber, an angular extension of which groove around the shared axis is larger than the angular extension of a through-opening extending through an entire thickness of the wall.

15. The device according to claim 14, wherein a center of the wall passage which forms an outlet for the first medium out of the sub-chamber located the furthest radially to the outside is axially offset vertically in relation to the mouth opening and/or the groove that is open toward the interior of the sub-chamber and is offset downwardly in the intended usage position.

16. The device according to claim 15, wherein the outside wall of the sub-chamber located the furthest radially to the outside forms an outer housing wall or at least a part of an outer housing wall of the entire device, the axial ends of which outer housing wall or part thereof comprise threaded regions, for the connection to wall regions of the device which enclose the gas compartments of the device through which the gas or gas mixture can be conducted to and from the interior of the hollow fibers.

17. The device according to claim 16, wherein one of the inside wall and the outside wall which delimit a sub-chamber is guided in the axial direction in a sealed manner through a gas compartment of the device and a wall region of the device which delimits the gas compartment from outside, a handle being attached or being attachable to a guided-through region by way of which the sub-chamber can be rotated about the shared axis or can be displaced along the shared axis.

18. The device according to claim 1, comprising only two sub-chambers.

19. The device according to claim 14, wherein an inside wall of the sub-chamber located radially the furthest to the inside forms a core of the device, which core is guided in the axial direction in a sealed manner through a wall region of the device which delimits a gas compartment, a handle being attached or being attachable to the core, by which the sub-chamber can be rotated about the shared axis or can be displaced along the shared axis.

20. The device according to claim 5, wherein a contact region of the outside wall and/or the inside wall of a sub-chamber in which the wall contacts a potting compound is coated with an adhesion promoter or is structured differently than a contact region of the wall in which the first medium is contacted, the other structuring being formed by a roughened region or by a region that is provided with grooves or recesses, or an annular, elastomeric sealing element resting at an axial end region of the outside wall and/or the inside wall of the sub-chamber in a notch, the sealing element being structured on the side thereof which contacts the potting compound and comprises grooves that extend in a circumferential direction.

21. The device according to claim 1, wherein the mass-permeable hollow fibers are disposed in at least one of a plurality of the sub-chambers and the heat-exchanging hollow fibers are disposed in at least one of a plurality of the sub-chambers for creating a heat exchanger function.

22. The device according to claim 1, wherein the device is configured to form an oxygenator for enriching blood as the first medium, with oxygen comprising the second medium, and/or to deplete carbon dioxide from blood as the first medium, the device comprising hollow fibers that are suitable for at least one of the aforementioned functions in at least some of the sub-chambers.
